(19) United States
(12) Patent Application Publication
(10) Pub. No.: US 2021/0375462 A1
(43) Pub. Date: Dec. 2, 2021

(54) SYSTEM AND METHOD UTILIZING SOFTWARE-ENABLED ARTIFICIAL INTELLIGENCE FOR HEALTH MONITORING AND MEDICAL DIAGNOSTICS

(71) Applicants: Jonathan C. Rayos, Sterling Heights, MI (US); Michael Angelo D'Orazio, Farmington Hills, MI (US)

(72) Inventors: Jonathan C. Rayos, Sterling Heights, MI (US); Michael Angelo D'Orazio, Farmington Hills, MI (US)

(21) Appl. No.: 17/337,142

(22) Filed: Jun. 2, 2021

Related U.S. Application Data
(60) Provisional application No. 63/033,698, filed on Jun. 2, 2020.

(51) Int. Cl. G16H 50/20; G06F 21/32; G06N 20/00; G16H 50/30; G16H 40/67; A61B 5/00; A61B 5/11; A61B 5/01; A61B 5/024; A61B 5/08; A61B 5/021; A61B 5/0205; G09B 5/02

(52) U.S. Cl. CPC G16H 50/20; G06F 8/30; G06N 20/00; G16H 50/30; G16H 40/67; A61B 5/7264; A61B 5/7275; A61B 5/1128; A61B 5/015; A61B 5/024; A61B 5/0816; A61B 5/021; A61B 5/02055; G09B 5/02; G06F 21/32

(57) ABSTRACT

The disclosed inventive concept provides a system and method incorporating extended reality, augmented reality, virtual reality, and artificial intelligence. The disclosed system and method utilizes these technologies to provide solutions to both the need for real-time medical diagnosis. Through the utilization of real-time 3D imaging, extended reality data, augmented reality data, and artificial intelligence, an actual real-time emergency or non-emergency medical response to an emergent or non-emergent medical condition provides immediate diagnostic information through predictive analytics from collective data to emergency medical services and hospital medical personnel. Through the utilization of real-time imaging through the use of telematics and high fidelity video cameras to help generate extended reality data, augmented reality data, artificial intelligence, and related software, an actual real-time medical response to both emergent and non-emergent situations is made possible by providing immediate diagnostic information through predictive analytics from collective data to emergency medical services and hospital medical personnel.

### TECHNICAL FIELD

The disclosed inventive concept relates generally to health monitoring and medical diagnostics. More particularly, the disclosed inventive concept relates to software-enabled artificial intelligence for use in the remote monitoring of the health of an individual and for providing medical diagnostics of the individual to a medical response team serving at a remote location. The disclosed inventive concept particularly resides in a software application driven by both artificial intelligence (AI) and machine learning (ML) and utilizes hardware within the ecosystem for delivery of data. The disclosed inventive concept more particularly resides in the utilization of Software-as-a-Medical Device (SaMD) that is enabled through artificial intelligence, machine learning, data code and cross reality (XR) for remote health and medical diagnostics monitoring in both emergency and non-emergency situations. The technical field encompasses use almost anywhere of real-time, enabled with artificial intelligence and machine learning through cloud-based databases of various individuals including those at home, in their place of business, in various industrial and agricultural settings, or in any mode of transportation.

### BACKGROUND OF THE INVENTION

The real-time assessment of an individual's vital physical condition in a variety of situations, both emergent and non-emergent, could provide first responders, emergency medical responders (EMR), emergency medical services (EMS), and emergency room (ER) hospital teams with the information needed on the condition of an individual to provide pre-diagnosis of a medical situation. While this is generally accepted as being a given, there exists a great disparity of healthcare workers in both rural and urban areas which leads to challenges in providing those with compromised health due to either illness or injury with real-time remote diagnostics and vital signs of in-home patients on a timely basis.

The impact of the lack of real-time information creates particular difficulties in the case of a transportation accident. Accidental automotive impact events are the leading cause of death in the United States for persons aged 1-54 with almost 40,000 people dying every year in vehicle accidents. Over four million people are injured annually in the United States in vehicle impact events seriously enough to require medical attention. Many of these fatalities could have been prevented and the injuries reduced if immediate medical attention was provided. However, known modes of transportation do not provide immediate real-time physical injury data to emergency medical services (EMS) personnel despite the fact that the time needed to gather diagnostics and vitals from occupants is crucial in emergency situations. Because transportation impact events today do not provide real-time physical injury data to emergency medical services (EMS) personnel, the ability to provide faster medical assistance is often compromised by time lost by medical personnel in diagnosing the scope of the actual injuries.

The interconnection of medical databases via the Internet using a distributed platform, namely the Internet of Things (IoT) which involves sensors, software, and related technologies to connect a variety of devices and systems, has the potential for providing needed real-time information on the condition of an individual However, the known arrangement lacks in collecting and combining key vitals and medical data from different sources in order to better diagnose patient health status and identify possible anticipatory actions.

The need for identifying different disease states remotely and in a prompt and complete manner became all the more critical with the onset of Covid-19 in late 2019. Certain features of the viral infection resulting from this virus, including specifically fever and changes in respiration, must be monitored to determine the likelihood of the individual having a viral infection.

Accordingly, there is a need to provide real-time information on the condition of an individual from any remote location to allow for the pre-diagnosis of the individual's state of health so as to enable early and timely treatment by medical personnel.

### SUMMARY OF THE INVENTION

The disclosed inventive concept overcomes the challenges faced by known medical responses by providing a system and method which benefits from advancements in the emerging broad area of the immersive technologies of extended reality (XR) in which physical and virtual worlds are merged. These technologies, including augmented reality (AR) and virtual reality (VR), focus on expanding the real world by various mechanisms, including the blending of both the virtual and the real world as well as formulating a completely immersive experience. In the case of augmented reality, the real world is modified by the use of virtual information and objects. This may involve the overlaying of virtual information and objects on elements of the real world whereby users are able to interact with the real world but in its modified or “augmented” form. Conversely, in virtual reality the user is immersed fully in a simulated digital environment. This area of technology is most often used by the gaming world but is becoming more common in other areas, such as in the healthcare industry as well as in the military.

The disclosed inventive concept involves primarily a software application driven by both artificial intelligence (AI) and machine learning (ML). The inventive concept utilizes hardware within the ecosystem for delivery of data. Beyond artificial intelligence and machine learning, the, disclosed inventive concept also relies on three-dimension (3D) imaging, and Motion-Capture (MoCap) visual data to provide immediate diagnostic information through predictive analytics followed immediately by the forwarding of collected data to emergency medical services (EMS), First-Responder personnel, and emergency room hospital medical personnel.

Through the utilization of real-time imaging through the use of telematics and high fidelity video cameras to help generate extended reality data, augmented reality data, artificial intelligence, and related software, an actual real-time medical response to both emergent and non-emergent situations is made possible by providing immediate diagnostic information through predictive analytics from collective data to emergency medical services (EMS) and hospital medical personnel.

Accordingly, the present inventive concept practically and effectively addresses the need for actual, real-time medical responses to both emergency and non-emergency events regardless of the location of the emergency. The present inventive concept achieves the needed real-time medical response by way of a variety of methods, including real-time three-dimension (3D), (XR), (AR), Motion-Capture (MoCap) visual data, and (AI). In addition, the application connects a (XR) and (AR) platform interface that helps train first responders and health professionals in all medical assessment situations. The application enhances (AR) and (AI) of telehealth data by remote diagnostics and monitoring in both emergency and non-emergency situations. The application utilizes (AI) in diagnosis, patient monitoring and care. The application is applied to enhance healthcare data management.

The inventive concept disclosed herein provides for a remote medical team to identify if not diagnose individuals having various viruses including but not limited to the Covid-19 virus. By remotely analyzing such vitals as the individual's body temperature and respiratory rate a view into the person's possible viral disease state may be assessed, thus providing a medical foundation for further assessment.

The above advantages and other advantages and features will be readily apparent from the following detailed description of the preferred embodiments when taken in connection with the accompanying drawings.

### BRIEF DESCRIPTION OF THE DRAWINGS

For a more complete understanding of this invention, reference should now be made to the embodiments illustrated in greater detail in the accompanying drawings and described below by way of examples of the invention wherein:

FIG. 1 is a flowchart illustrating the operation of the disclosed inventive concept;

FIG. 2 is block diagram illustrating in detail the initial step of image capturing and user identity validation including hardware used in this step;

FIG. 3 is block diagram illustrating in detail the step of engaging user groups according to the different situation and performing diagnostics including hardware used in this step;

FIG. 4 is block diagram illustrating in detail the step of engaging machine learning/artificial intelligence to make physical assessments and a determination of primary medical vitals;

FIG. 5 is block diagram illustrating in detail the different body components assessed for diagnosis and treatment; and

FIG. 6 is block diagram illustrating in detail the steps of detecting viral infections such as the Covid-19 virus and assessing dynamic skin responses including accessing appropriate databases.

### DETAILED DESCRIPTION OF THE PREFERRED EMBODIMENTS

In the following figures, the same reference numerals will be used to refer to the same components. In the following description, various operating parameters and components are described for different constructed embodiments. These specific parameters and components are included as examples and are not meant to be limiting.

The system incorporates pre-installed databases, analysis tool programs, and interpretive software including programs for applying an enhanced reality program to the captured images of individuals in a variety of remote circumstances. The interpretive software programs interpret images received by image capturing devices in proximity of the individual, whether in a fixed structure such as a home or business or in a mobile unit such as a motor vehicle. The interpretive software program utilizes extended reality, enhanced augmented reality, artificial intelligence, and machine learning datasets to interpret any physical condition in real time and provides an analysis of the injury and a recommended course of treatment.

When operating in its injury analysis and recommended treatment mode, the software summarizes, organizes, and manages diagnostic data. The diagnostic data may be organized in a specific format, such as assessing and recommending treatment of an injury to a specific internal organ. The software program further enables the data related to the identification and extent of the specific physical condition as well as a recommended course of treatment to be transmitted from the local network integrated with the imaging system to a remotely located server for use by medical personnel. The preloaded software may include application programs and analysis tool programs.

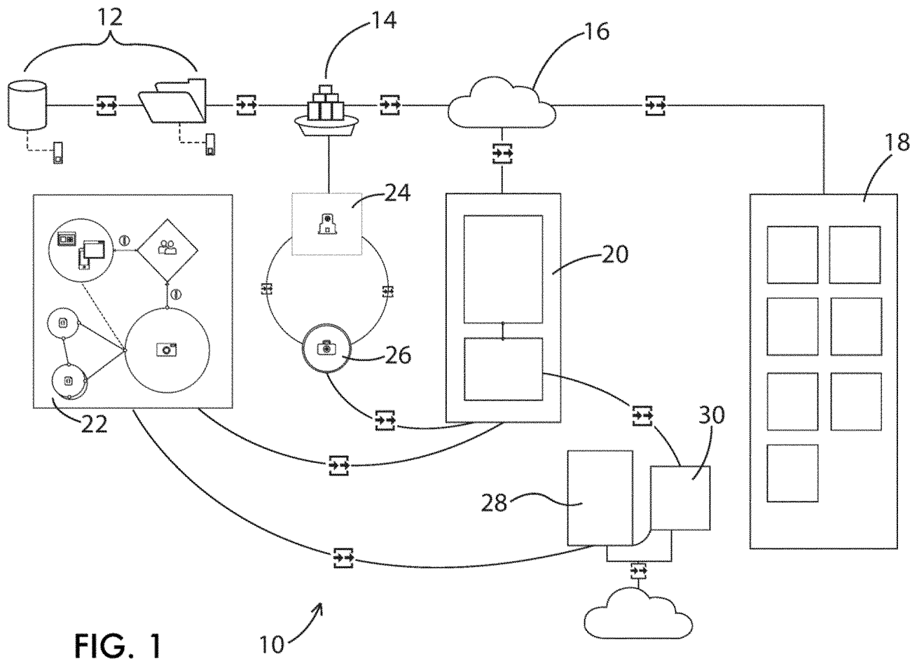

Referring to FIG. 1 a flowchart illustrating the operation of the disclosed inventive concept is shown. It is to be understood that the illustrated flowchart is to be considered as a preferred arrangement but not an exclusive arrangement as it is possible that one or more changes may be made to the flowchart without deviating from the scope of the invention as described.

The operational format of the disclosed inventive concept, generally illustrated as 10, includes the necessary software application, the Software-as-a-Medical Device (SaMD) application, needed to assist emergency first-responders, medical professionals, hospital emergency personnel to assess and diagnose in real-time—through extended reality (XR), Motion-Capture (MoCap) visual data anchored by deep machine learning (ML), and artificial intelligence (AI). The operational format 10 provides a pathway with all of the necessary hardware to enable remote monitoring and diagnosis for health and medical situations thereby assisting in emergency responsiveness for health and medical situations.

The operational format 10 includes discrete steps between the initial input of data to the diagnosis and proposed treatment regimen for a variety of illnesses and disease states. A patient/user database 12 is provided comprising, for example, a magnetic data storage unit and electronic folders. The collected data is inputted/outputted to a data center 14 including appropriate servers. A code file 16 provides a predictive analysis of the health condition of the individual being assessed. The predictive analysis is based on inputs received from the individual's observed physical evidence 18 and generated by learning/artificial intelligence 20. The learning/artificial intelligence 20 including making both physical assessments and determining primary medical vitals receives inputs from captured information 22 once the identity of the user is validated, identified user groups 24 and requisite user group diagnostics 26, detection of early vital detection 28, and dynamic skin responses 30. The latter two, early vital detection 28 and dynamic skin response 30, are preferably related to certain conditions such as, but not limited to, viral conditions caused by, for example, the Covid-19 virus. It is to be understood that other specific conditions beyond Covid-19 may be sensed including other viral conditions and further including bacterial conditions.

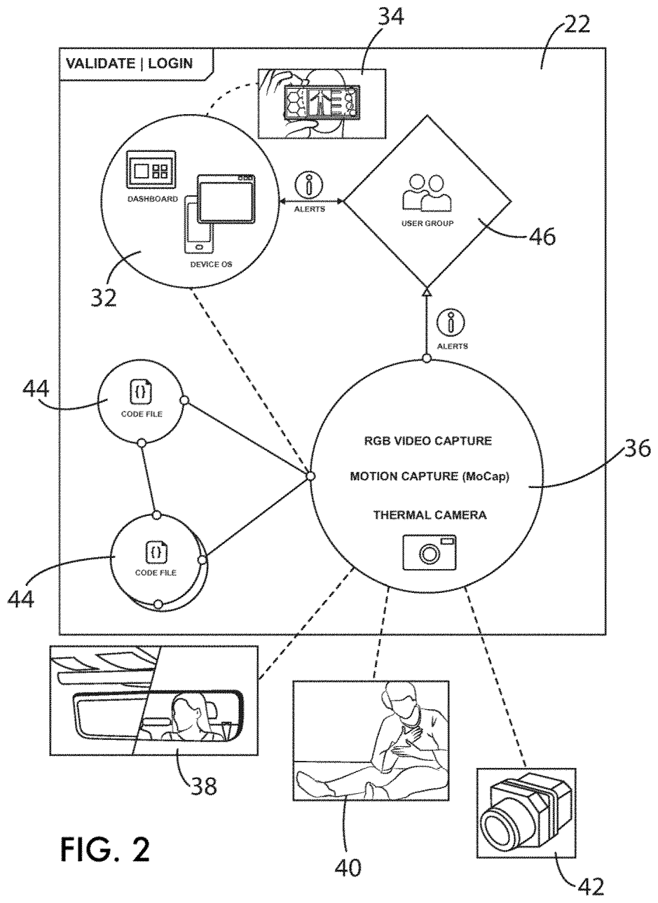

Referring to FIG. 2, the systems for capturing captured information 22 and for communicating this information with the system for learning/artificial intelligence 20 are illustrated in detail. The captured information 22 is gathered and relayed preferably though not necessarily as alerts generated by personal communication devices 32 such as but not limited to a mobile device, a personal computer, or a fixed or mobile workstation. More particularly, utilization and login may be made from any acceptable technology device having access via 4G, 5G, or WiFi networks and from iOS mobile or Android mobile devices. A personal communication device such as a cell phone 34 may also be used for this purpose.

In addition, images disclosing the individual's condition may be made by image capturing devices 36 by an application that initiates and captures visual content in video file format from, for example, a motion capturing device (MoCap), a video capture by way of, for example, a 2K/4K video camera, or by way of a thermographic camera. Such devices may be used alone or in conjunction with one another as required to perform the operation. Inputs are specifically provided from sources such as, but not limited to, in-vehicle imaging equipment 38, in-home/in-office imaging equipment 40, and various cameras 42.

The image capturing application enables real-time data by augmented-intelligence, utilizing telematics for medical assessments, and diagnosis of transportation occupants in an emergency or non-emergency situation. The application enhances augmented-intelligence of telehealth data by remote diagnostics and monitoring in both emergency and non-emergency situations. The application will use at times, the thermographic camera used for the capture of enhanced thermal imaging using infrared to access an the temperature of an object or an individual. The application utilizes a mobile application to deliver and collect multiple diagnostic images and health vital signs.

The images captured by the image capturing devices 36 are stored in code files 44 for later reference or for further processing. The code files 44 may be of any type ordinarily used for this purpose.

Alerts or other messaging generated by the personal communication devices 32 and the image capturing devices 36 are delivered to validated users or user groups 46 by way of a cross reality (XR) and augmented reality (AR) platform interface that not only delivers vital information as to the status of the health of an individual but also assists in the training of first responders and health professionals in all medical assessment situations.

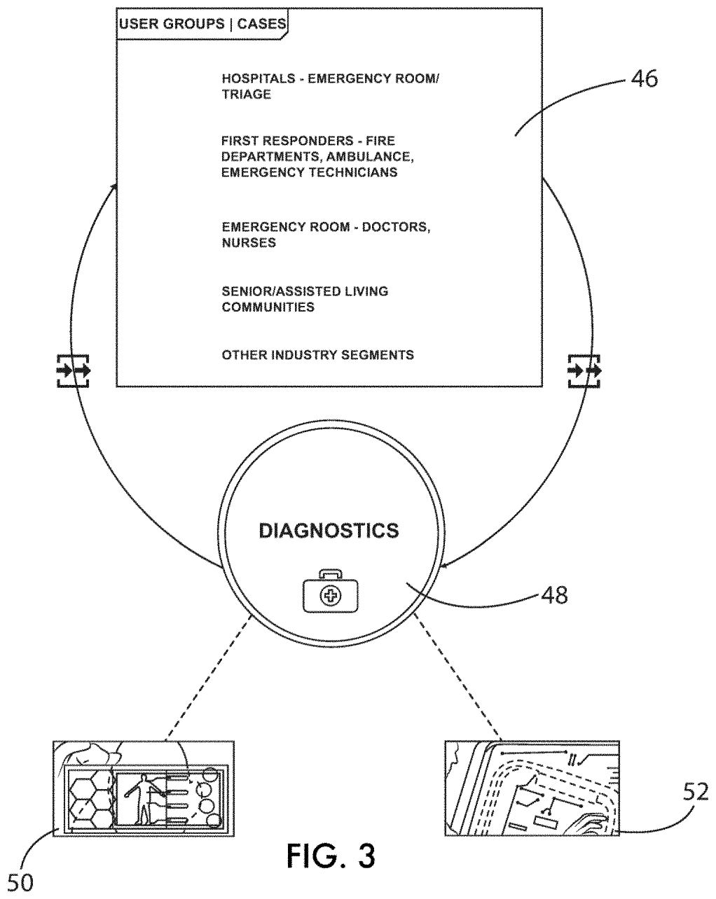

Non-limiting examples of users and user groups 46 are illustrated in FIG. 3. With reference thereto, the users and user groups may include hospitals-emergency room triage, first responders (fire departments, ambulance services, emergency room healthcare providers (doctors, nurses), healthcare professionals in senior or assisted living communities, as well as other segments of the healthcare community.

Specifically, the application provides diagnostic information 48 to first responders, emergency medical services (EMS), and emergency room (ER) hospitals real-time pre-diagnostics on key vital data, artificial intelligence (AI) and predictive analysis of physical injuries on medical situations. Using the application, collected multiple physical diagnostic images and key health vital signs from imaging equipment 50 are delivered through a mobile application. The application uses fully interactive augmented reality (AR) and cross reality (XR) diagnostics 52 using images which are transmitted to medical care. Artificial intelligence of the application can be applied to health care interventions and patient care. The application connects a cross reality (XR) and an augmented reality (AR) platform interface to help in the above-mentioned training if first responders and health professionals in all medical assessment situations.

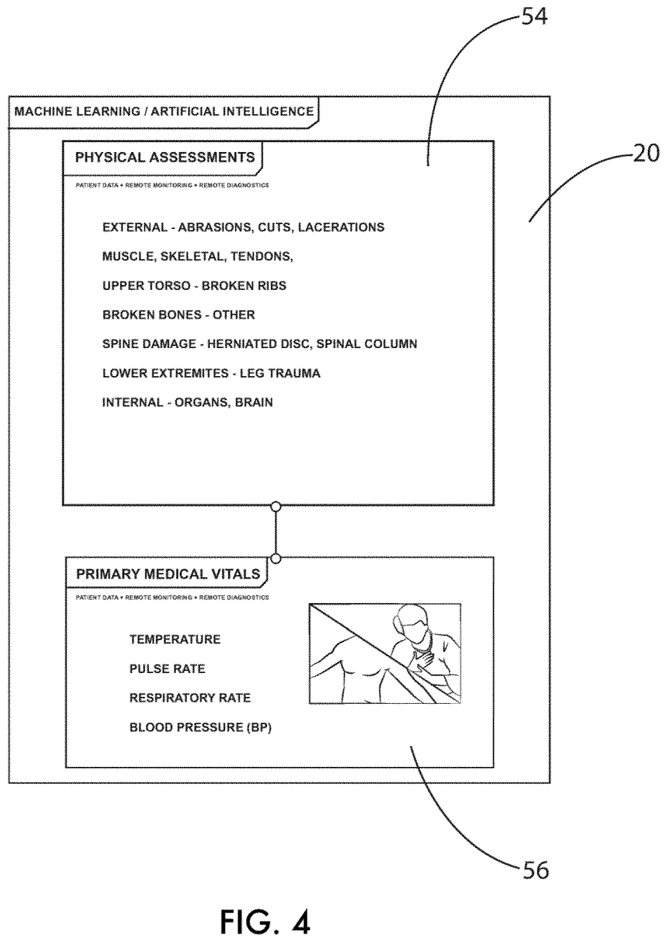

Referring to FIG. 4, and as stated, the application utilizes learning/artificial intelligence 20 in diagnosis, patient monitoring and care based on information generated by the captured information 22. The application is applied to enhance healthcare data management and utilizes an artificial intelligence algorithm to analyze and learn useful standards from clinical datasets to thereby provide better evidence to support the decisions of health professionals and thus help to improve patient health outcomes in hospitals. The application gathers visual augmented reality analysis of real-time pre-diagnostics on key vital data, artificial intelligence and predicted analysis of physical injuries on medical situations. More particularly, and as noted, the application enhances augmented-intelligence of telehealth data by remote diagnostics and monitoring in both emergency and non-emergency situations.

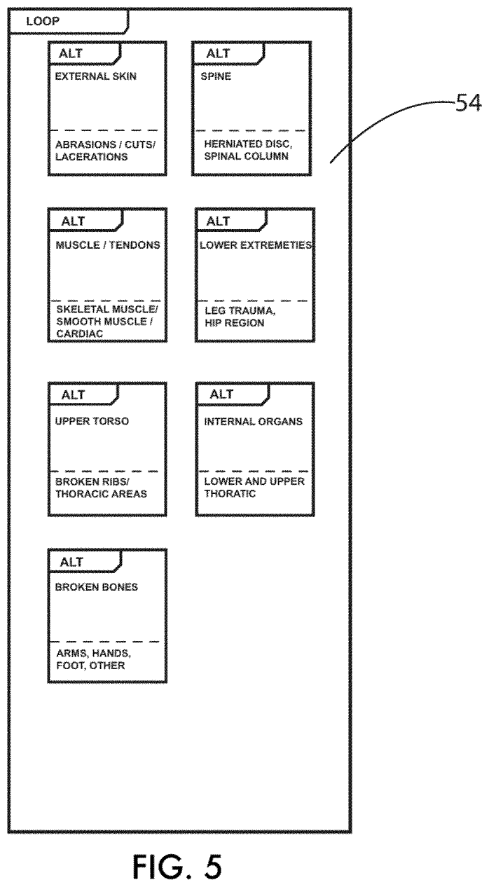

The information generated by the captured information 22 is inputted to the learning/artificial intelligence 20. A variety of physical assessments 54 may be made visually to thereby determine specific conditions. As non-limiting examples, assessments are made of any external injuries (abrasions, cuts, lacerations) muscle damage (skeletal, tendons), injury to the upper torso (for example, broken ribs), broken bones that may be visualized, damage to the spine that may be visualized (herniated disc, spinal column injury), damage to the lower extremities (for example, leg trauma), or internal injury (internal organs, brain damage) that may be visualized. These specific physical assessments 54 are also set forth in FIG. 5 which identifies the variety of areas of the body subject to visual characterization.

Additional information that may be generated and communicated includes primary medical vitals 56 such as but not limited to temperature, pulse rate, respiratory rate, and blood pressure.

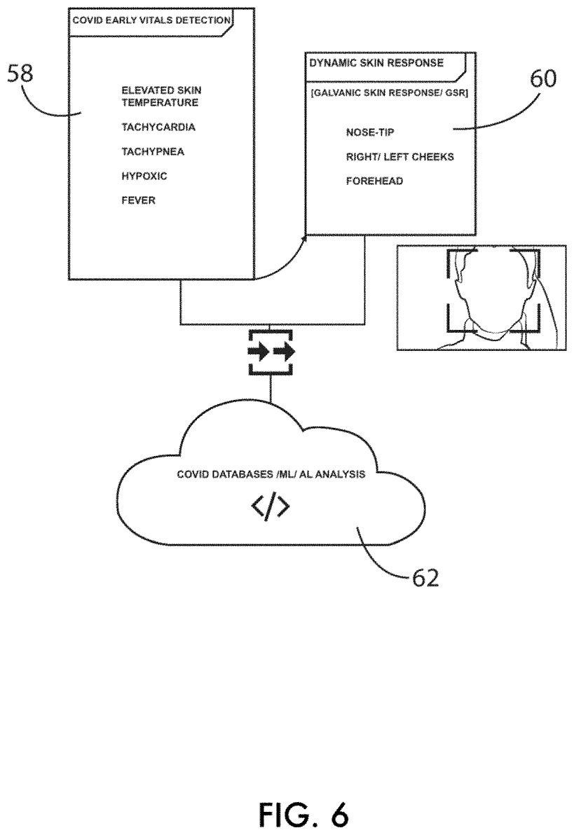

The system of the disclosed inventive concept finds particular usefulness in the diagnosis of particular disease states. Importantly, the disclosed system may be adjusted for a given disease state. By way of example, and as illustrated in FIG. 6, the disclosed inventive concept is useful in the early detection of viral infections such as that caused by the Covid-19 virus. The application of the disclosed system is able to monitor vital signs 58 for the early detection of Covid-19. Particularly, the application collects medical vitals including an individual's temperature, pulse rate, respiratory rate, and blood pressure and may, in particular, detect elevated skin temperature, tachycardia, tachypnea, hypoxia, and elevated temperature.

The system of the disclosed inventive concept further includes an application to detect dynamic skin temperature through galvanic skin response (GSR) 60. The appropriate analytics for this step may be collected from the nose-tip, the right/left cheeks, and the forehead.

Information derived from the monitoring of vital signs 58 and from the galvanic skin response 60 is processed in an appropriate database using, for example, a natural language processing and data analytics code specific to Covid-19 health care and relying upon artificial intelligence (AI) code to extract value added outcomes from all known Covid-19 medical cloud sources 62. The application will be addressed in real-time, through artificial intelligence (AI) and deep machine learning (ML) code.

The information gathered provided by the captured information 22, the identified user groups 24, the user group diagnostics 26, the early detection 28, and the dynamic skin response 30 is provided to the code file 16 to generate a predictive analysis of the assessed individual's condition.

As set forth above, the images generated according to the disclosed system and method are usable in a broad variety of applications including, but not limited to, use by medical support services for assessing the medical condition of an individual to provide more timely and more successful medical treatment. One skilled in the art will readily recognize from such discussion, and from the accompanying drawings and claims that various changes, modifications and variations can be made therein without departing from the true spirit and fair scope of the invention as defined by the following claims.

What is claimed is:

1. A method to provide remote medical assessment of an individual comprising:
forming a software-as-a-medical device application capable of helping emergency first-responders, medical professionals, hospital emergency personnel assess and diagnose in real-time, the application enabling real-time data by augmented intelligence and utilizes telematics for medical assessment and diagnosis of the individual, the application utilizing an artificial intelligence algorithm to analyze and learn standards from clinical datasets to support medical decisions, the application using natural language processing and data analytics code specific to health care
forming an image capturing device for capturing the image of an individual, the images being interactive augmented reality and cross reality images;
gathering visual augmented reality, extended reality, and virtual reality analysis of real-time pre-diagnostics on vital date, artificial intelligence, and predicted analysis of physical injuries;
using the application to provide predictive health analytics based on the assessment of the individual's physical condition; and
relaying the predictive analytics to a user group.

2. The method of claim 1 wherein the software-as-a-medical device application validates users by utilization and login from a specified device.

3. The method of claim 1 wherein the image capturing device captures visual content of a video file format from a camera and initializes motion capture visual data points.

4. The method of claim 1 wherein the image capturing device is a thermographic camera for capturing enhanced thermal imaging using infrared visualization thereby enabling the determination of the individual's temperature, pulse rate, respiratory rate, and blood pressure.

5. The method of claim 1 wherein the application collects medical vital data from the gathered images, the vital data including temperature, pulse rate, respiratory rate, and blood pressure.

6. The method of claim 1 wherein the application detects conditions selected from the group consisting of elevated skin temperature, tachycardia, tachypnea, hypoxia, and elevated fever.

7. The method of claim 1 wherein the application includes a processing and analytics code specific to the care of a virus.

8. The method of claim 7 wherein said virus is the Covid-19 virus.

9. A method to assist in the training of remote medical personnel in the assessment of an individual comprising:
forming a software-as-a-medical device application capable of training emergency first-responders, medical professionals, hospital emergency personnel in the assessment and real-time diagnosis of the health status of an individual, the training being aided through the connection by the application to an augmented reality and cross reality platform training interface;
forming an image capturing device for capturing the image of an individual;
gathering visual augmented reality analysis of real-time pre-diagnostics on vital date, artificial intelligence, and predicted analysis of physical injuries;
using the application to provide predictive health analytics based on the assessment of the individual's physical condition; and
relaying the predictive analytics to the remote medical personnel for training.

10. A method to provide remote medical assessment of an individual comprising:
forming at least one image capturing device;
placing said image capturing device in proximity of an individual to be assessed;
forming a system of conveying the captured information to a program for assessing the condition of the individual;
forming software-as-a-medical device application to analyze the conveyed captured information, the application being enabled through artificial intelligence, machine learning, data code, and cross reality to assess the physical condition of an individual in real time;
forming a system for providing predictive health analytics based on the assessment of the individual's physical condition; and
relaying the predictive analytics to a user group.

11. The method of claim 10 wherein said system of conveying captured information is taken from the group consisting of a mobile device, a personal computer, or a fixed or mobile workstation.

12. The method of claim 10 wherein said system of conveying captured information has network access via 4G, 5G, or WiFi networks and from iOS mobile or Android mobile devices.

13. The method of claim 10 where the user group is alerted to incoming information related to the physical condition of the individual.

14. The method of claim 10 where the user group includes healthcare professionals.

15. The method of claim 10 wherein the assessment of the individual includes possible external and internal injuries.

16. The method of claim 15 wherein specific assessment is made of externally apparent injuries.

17. The method of claim 15 wherein primary medical vitals are taken including measurements of the individual's temperature, pulse rate, respiratory rate, and blood pressure.

18. The method of claim 15 wherein detection of possible viral infection is made through determination of one or more vital signs selected from the group consisting of elevated skin temperature, tachycardia, tachypnea, hypoxia, and fever.

19. The method of claim 10 wherein the application includes virtual reality, artificial intelligence, and predictive analytics.

20. The method of claim 10 wherein the predictive analytics are directed to a medical treatment unit.

21. A system for providing remote medical assessment of an individual, the system comprising:
a software-as-a-medical device application capable of helping emergency first-responders, medical professionals, hospital emergency personnel assess and diagnose in real-time, the application enabling real-time data by augmented intelligence and utilizes telematics for medical assessment and diagnosis of the individual, the application utilizing an artificial intelligence algorithm to analyze and learn standards from clinical datasets to support medical decisions, the application using natural language processing and data analytics code specific to health care
an image capturing device for capturing the image of an individual, the images being interactive augmented reality and cross reality images; and
a relay system for remotely relaying visual augmented reality, extended reality, and virtual reality analysis of real-time pre-diagnostics on vital date, artificial intelligence, and predicted analysis of physical injuries and predictive health analytics based on the assessment of the individual's physical condition to a user group.
